(19) United States
(12) Patent Application Publication
(10) Pub. No.: US 2024/0008895 A1
(43) Pub. Date: Jan. 11, 2024

(54) NEEDLE GUIDANCE SYSTEM

(71) Applicant: THE REGENTS OF THE UNIVERSITY OF COLORADO, A BODY CORPORATE, Denver, CO (US)

(72) Inventors: Land Belenky, Denver, CO (US); John Lemery, Denver, CO (US)

(73) Assignee: THE REGENTS OF THE UNIVESITY OF COLORADO, A BODY CORPORATE, Denver, CO (US)

(21) Appl. No.: 18/255,854

(22) PCT Filed: Dec. 8, 2021
(86) PCT No.: PCT/US21/62488

§ 371 Date: Jun. 2, 2023

Related U.S. Application Data
(60) Provisional application No. 63/122,600, filed on Dec. 8, 2020.

(51) Int. Cl. A61B 17/34

(52) U.S. Cl. CPC A61B 17/3403; A61B 2017/3413

(57) ABSTRACT

Guidance systems and methods for placing a needle in a body are disclosed. Exemplary systems can be used to independently manipulate a probe transducer and a needle guide to determine an anticipated path of the needle within the body.

### CROSS REFERENCE TO RELATED APPLICATIONS

This application claims the benefit of U.S. Provisional Application Ser. No. 63/122,600 filed Dec. 8, 2020, entitled “NEEDLE GUIDANCE SYSTEM”, the contents of which are hereby incorporated herein by reference, to the extent such contents do not conflict with the present disclosure.

### BACKGROUND

For years ultrasound transducers have been used to position and place needles. Conventional systems generally take one of two forms: either the needle is passed through a needle guide rigidly attached to the ultrasound probe in order to set the position of the needle in a known/controlled orientation or the needle is detected by the ultrasound probe and displayed in the sonogram. Conventional systems/techniques have limitations and shortcomings.

If using a probe with a rigidly attached needle guide, it's critical that the position of the needle guide is carefully calibrated and does not shift because the placement of the needle is only as good as the external guide. The angle of approach and position of the needle is limited. Also, the movement of the ultrasound probe is restricted during insertion, which may inhibit the ability to obtain a proper sonogram image. The challenge of detecting the needle via the ultrasound transducer is that the ultrasound may not sufficiently detect the presence of the needle. Further, the position of the needle cannot be determined until after it is inserted into the body. In some conventional arrangements, the needle is not visible at all until it intersects the image plane, and even then it may go in and out of visibility as the probe is moved or turned. Additionally, some types of needles show very little image on the screen, or sometimes the shaft of the needle can be seen, but the tip does not appear or vice versa. While there are various conventional methods to improve imaging of the needle, such as filling it with air or water, using a larger bore needle, roughening the surface, or wiggling it while it is in the patient, none of these are truly reliable and all have drawbacks and shortcomings.

### SUMMARY OF INVENTION

Exemplary embodiments of this disclosure provide a system and method for guiding a needle into a body.

In various embodiments, a needle guidance system comprises a probe comprising a probe transducer and a camera; and a needle guide configured to retain a needle, wherein the needle guide comprises a plurality of fiducials. In various embodiments, the plurality of fiducials comprises at least four fiducials. In various embodiments, the probe comprises an ultrasound probe. In various embodiments, the probe transducer is an ultrasound transceiver configured to transmit and receive ultrasound.

In various embodiments, each fiducial of the plurality of fiducials has a characteristic that is unique from other fiducials of the plurality of fiducials. In various embodiments, each fiducial of the plurality of fiducials comprises a color that is different from other fiducials of the plurality of fiducials. In various embodiments, each fiducial of the plurality of fiducials comprises a shape that is different from other fiducials of the plurality of fiducials. In various embodiments, the system uses a heuristic calculation to distinguish each fiducial from the other fiducials of the plurality of fiducials.

In various embodiments, the camera is a wide-angle camera. In various embodiments, the camera comprises two cameras, and the plurality of fiducials comprises three fiducials.

In various embodiments, the probe and the needle guide are configured to be manipulated independently of each other.

In various embodiments, a method of providing position information of a needle comprises positioning a needle guidance system; the system comprising a probe comprising a probe transducer and a camera, and a needle guide comprising the needle and a plurality of fiducials; using the camera, obtaining a first image of the plurality of fiducials; transmitting the first image to a computing device; using the computing device, calculating the position information of the needle; using the probe transducer, obtaining a second image; transmitting the second image to the computing device; using the computing device, combining the second image with the position information; and displaying the second image with the position information on an output device. In various embodiments, the position information comprises the position and orientation of the needle relative to the probe.

In various embodiments, the probe comprises an ultrasound probe, and the ultrasound probe is positioned against a body. In various embodiments, the probe transducer is an ultrasound transceiver configured to transmit and receive ultrasound. In various embodiments, the computing device is further configured to calculate a trajectory of the needle.

In various embodiments, the plurality of fiducials comprises at least four fiducials. In various embodiments, each of the plurality of fiducials has a characteristic that is unique from the other of the plurality of fiducials. In various embodiments, the camera is a wide-angle camera. In various embodiments, the camera comprises two cameras, and wherein the plurality of fiducials comprises three fiducials.

### BRIEF DESCRIPTION OF THE DRAWINGS

FIG. 1 is a schematic depiction of a needle guidance system, in accordance with various embodiments;

FIG. 2 is a schematic depiction of a probe of the needle guidance system, in accordance with various embodiments;

FIG. 3 is a schematic depiction of another implementation of the probe of the needle guidance system, in accordance with various embodiments;

FIG. 4 is a schematic depiction of a needle guide of the needle guidance system, in accordance with various embodiments;

FIG. 5A is a schematic depiction of the needle guidance system in operation, in accordance with various embodiments;

FIG. 5B is a schematic depiction of the needle guidance system in operation, in accordance with various embodiments;

FIG. 6A is a schematic depiction of the various possible orientations of the needle guide if only one fiducial was utilized to determine the position of the needle guide, in accordance with various embodiments;

FIG. 6B is a schematic depiction of the various possible orientations of the needle guide if only two fiducials were utilized to determine the position of the needle guide, in accordance with various embodiments;

FIG. 6C is a schematic depiction of the various possible orientations of the needle guide if only three fiducials (and a single camera) were utilized to determine the position of the needle guide, in accordance with various embodiments; and

FIG. 7 illustrates a method in accordance with various embodiments.

The subject matter of the present disclosure is particularly pointed out and distinctly claimed in the concluding portion of the specification. A more complete understanding of the present disclosure, however, may best be obtained by referring to the detailed description and claims when considered in connection with the drawing figures, wherein like numerals denote like elements.

### DETAILED DESCRIPTION

The detailed description of exemplary embodiments herein makes reference to the accompanying drawings, which show exemplary embodiments by way of illustration. While these exemplary embodiments are described in sufficient detail to enable those skilled in the art to practice the disclosure, it should be understood that other embodiments may be realized and that logical changes and adaptations in design and construction may be made in accordance with this disclosure and the teachings herein without departing from the spirit and scope of the disclosure. Thus, the detailed description herein is presented for purposes of illustration only and not of limitation.

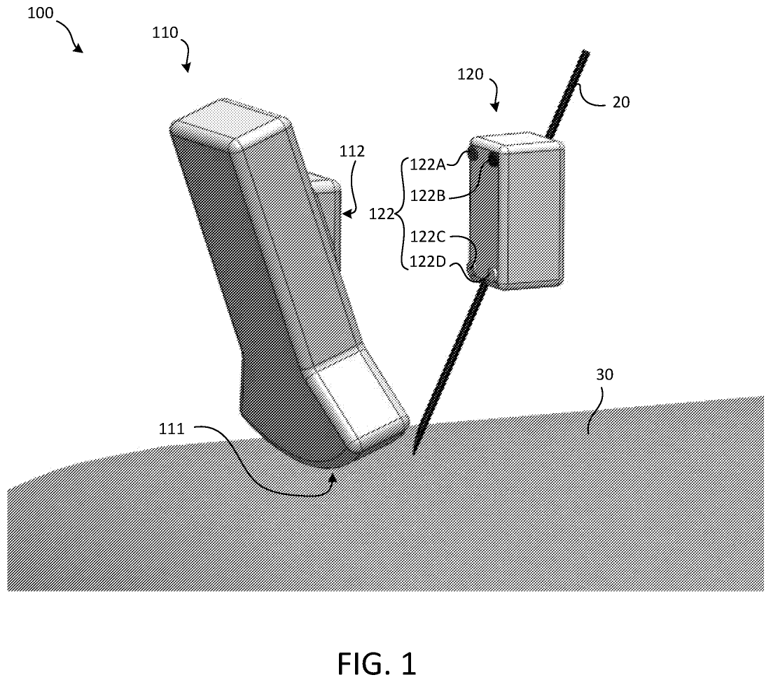

In various embodiments, and with reference to FIG. 1, a needle guidance system 100 is provided. The needle guidance system 100 generally includes a probe 110 and a needle guide 120 coupled to or configured to retain a needle 20. The needle guidance system 100 generally provides (1) the ability to independently manipulate the needle 20 while also independently manipulating the probe 110 and (2) the ability to determine the position of and project the anticipated path of the needle 20 before the needle 20 enters the body 30. Generally, a camera 112 is coupled to, mounted to, or otherwise attached to the probe 110, and the camera 112 is configured to detect a plurality of fiducials 122. For example, the plurality of fiducials may include four fiducials 122A, 122B, 122C, and 122D (collectively, fiducials 122) of the needle guide 120. As described in greater detail below, detection of the fiducials 122 by the camera 112 enables and allows the position and orientation of the needle guide 120 to be determined/calculated, which is indicative of the position and orientation of the retained needle 20. In various embodiments, the camera 112 is not positioned or disposed separate from the probe 110, but instead is directly mounted to the probe 110.

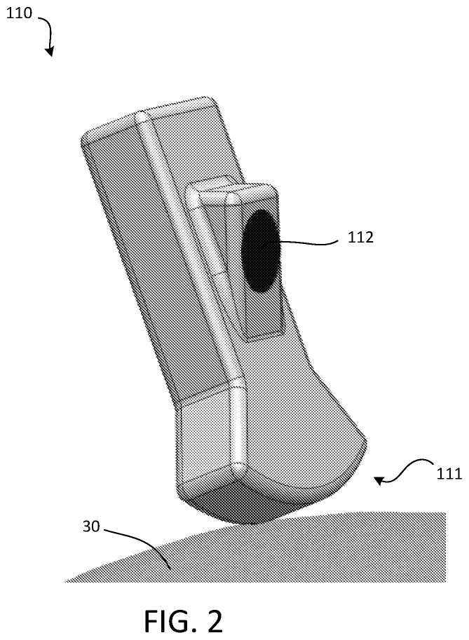

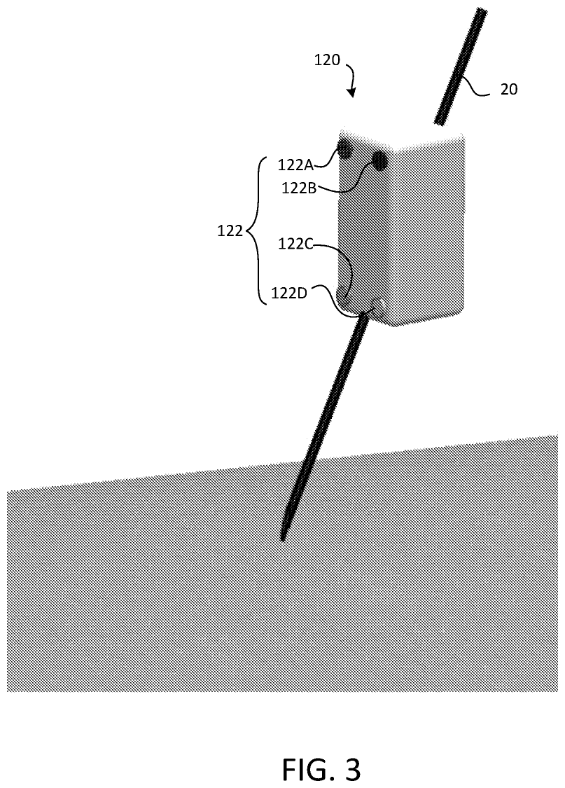

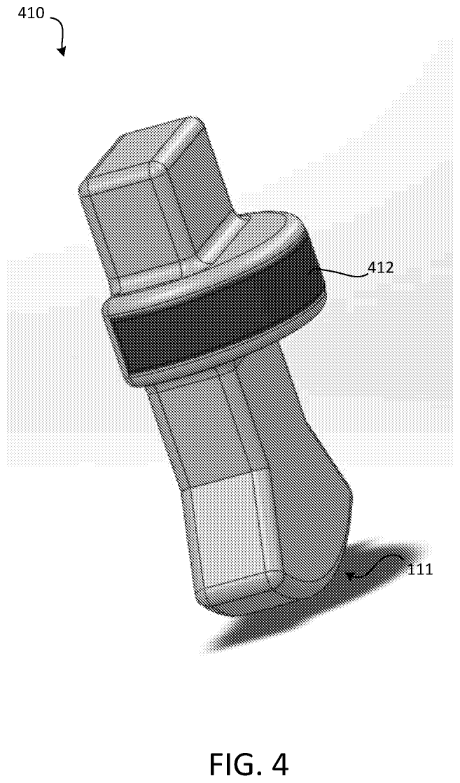

In various embodiments, and with reference to FIGS. 1, 2, and 3, the probe 110 includes a probe transducer 111, such as an ultrasound transducer, and a camera 112. The probe 110 may be a component of an imaging assembly, such as an ultrasound imaging assembly, and may include a corresponding probe transducer 111. While numerous details are included herein pertaining to ultrasound probes and sonogram images, other types of probes may be implemented in the needle guidance system 100. In various embodiments, the probe transducer 111 is an ultrasound transceiver that both transmits and receives ultrasound. The camera 112 may be coupled to or integrally formed with the probe 110. Additional details relating to the camera 112 are included below with reference to FIG. 4.

The needle guide 120, according to various embodiments and with continued reference to FIGS. 1, 2, and 3, is coupled to or is otherwise configured to retain the needle 20. For example, the needle guide 120 with its fiducials 122, which are described in greater detail below, may be repeatedly used to hold different needles, and thus may be detachably coupled to the needle 20. In an example embodiment, the position and orientation of the needle 20 relative to the needle guide 120 known/fixed. In various embodiments, the needle guide 120 may be a portion, a segment, or a section of the needle 20 itself (e.g., formed on a portion of the needle 20 that remains visible to the camera 112). That is, in an example embodiment, the fiducials 122 of the needle guide 120 may be formed on the surface of the needle 20 itself. As used herein, the term “needle” refers generally to devices or objects that are used to puncture or lacerate the skin (e.g., sharps), and thus may include hypodermic needles, scalpels, blades, etc.

In various embodiments, the camera 112 is mounted on the body of the ultrasound probe 110 and the camera 112 may be configured to obtain an image (s) of the markings/fiducials 122 on the needle guide 120. The image is then transmitted to a computing device, such as a computer or a controller with a processor, that is configured to calculate the relative position and orientation of the needle guide 120 (and thus the needle 20) relative to the ultrasound probe 110. The computing device may further combine the ultrasound image (e.g., sonogram) with the position and path of the needle 20 and display it to the user (e.g., operator or practitioner). In various embodiments, the camera 112 is configured to generally face towards the space where the needle guide 120 and needle 20 will be utilized. In various embodiments, the orientation of the camera 112 may be customized/adjusted, and the corresponding calculations by the computing device may take into account the adjusted position of the camera 112.

In various embodiments, the fiducials 122 are colored, which is helpful for describing the algorithm and may be helpful in operation, but is not strictly necessary. That is, the fiducials 122 do not have to be distinguished from each other by color, the fiducials 122 may be distinguished by shape (e.g., square, circle, diamond, triangle, etc) or may have other unique or distinguishing features, or may be indistinguishable other than by position. The size, shape, color or pattern of the fiducials 122 may be used to indicate the size and type of the needle 20, or a separate marking on the needle guide 120 could convey this information. As used herein, the term “fiducial” means a visible marking which the camera and computer are able to mathematically associate with a single position datum. As described in greater detail below, in one example embodiment, completely and accurately determining the position and orientation of the needle guide 120 requires data from multiple fiducials 122 being read, imaged, and/or detected, simultaneously. In various embodiments, each fiducial may be a more complicated visual mark (i.e., may be more than a single point that provides a single position datum). For example, a triangular mark with three visually identifiable corners may be three fiducials (one for each corner). Similarly, a circular mark, of which one may read/identify both the position and the diameter of the circular mark, may be considered two fiducials because it conveys two pieces of information. In various embodiments, a heuristic method may be utilized to resolve the position of the needle guide 120.

In various embodiments, and with reference to FIG. 4, a wide-angle camera 412 may be utilized on the probe 410. The wide-angle camera 412 may wrap around one or both edges/ends of the probe 410 so the operator can rotate the probe 410 for in-plane and out-of-plane imaging while keeping the needle guide 120 within the visual range of the camera 412. Such a configuration may be accomplished with a single wide-angle camera (as shown), a fish-eye camera (such as in the Garmin VIRB 360), two or more separate cameras mounted at different angles, or one camera that either moves side-to-side or records an image from a mirror that moves side-to-side.

In various embodiments, and returning to reference FIGS. 1, 2, and 3, the camera 112 may be sealed to the body of the probe 110 to prevent water ingress, and the housing of the camera 112, as well as the entire needle guidance system 100, may be configured to satisfy tests of bio-compatibility, cytotoxicity, sterilizability, and mechanical durability, among others. In various embodiments, the camera 112 may have a focal distance of between about 10-30 centimeters, an angular field of view of about 60 degrees, a pixel resolution of at least 1920×1080, and a refresh rate of 30 to 60 Hz (such details are merely exemplary/illustrative, and thus the scope of the present disclosure is not limited by such details). While certain conventional systems may utilize reflectors and/or light emitting diodes disposed on a needle retainer, such conventional systems rely on reflection and are thus constrained to only work when the relative position of the probe and needle retainer fulfill the condition that the angle of incidence equals the angle of reflection. Thus, in various embodiments, the fiducials 122 do not comprise transponders or electro-optical sensors.

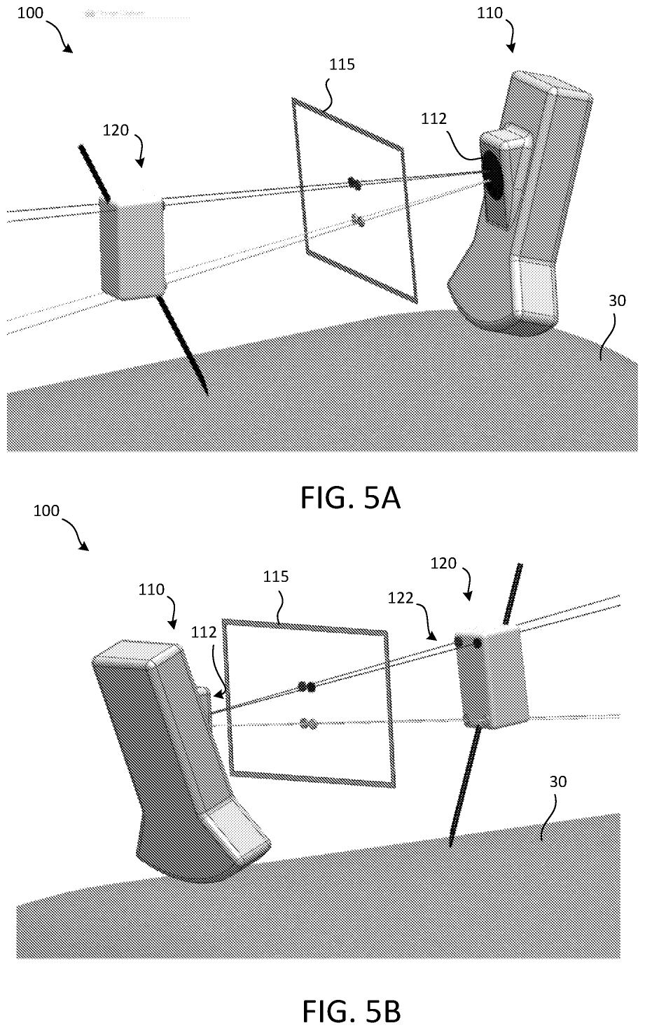

In various embodiments, and with reference to FIGS. 5A and 5B, illustration of the needle guidance system 100 in operation is provided. The distance between the probe 110 and the needle guide 120 may not be indicative of an actual use scenario, but the relative positions are shown as such for clarity of the figure. An imaginary image plane 115 is shown in FIGS. 5A and 5B with points corresponding to the four fiducials 122. The depicted rays correspond to the fiducials 122 and the points on the imaginary image plane 115, and are representations of how the algorithm of the computing device calculates the position of the needle guide 120. That is, the camera 112 produces an image of these fiducials 122 by essentially mapping them back along a straight line from the true position of the fiducials 122 to the image plane 115. In various embodiments, the positions of the points in the imaginary image plane 115 may be converted to spherical coordinates. In various embodiments, the position of the needle guide 120 is determined from the apparent positions of the fiducials on the image plane using the linear algebra of matrix transformations for rotation and translation in three dimensions.

The X and Y position of the points in the imaginary image plane 115 can be determined. However, because the camera 112 produces a flat image, information about the Z direction may not be readily apparent. Therefore, the computing device is configured to deduce the precise position and orientation of the needle guide 120 using only the coordinates of the four pixels that correspond with the four fiducials 122 and the known geometry of the camera 112 and the known horizontal and vertical spacing the fiducials 122 on the needle guide 120. While another approach would be to use two cameras and three fiducials (or just two fiducials if they were coaxial with the needle), the present disclosure describes the calculations for a system that includes a single camera 112 on the probe 110 and four fiducials 122 on the needle guide 120.

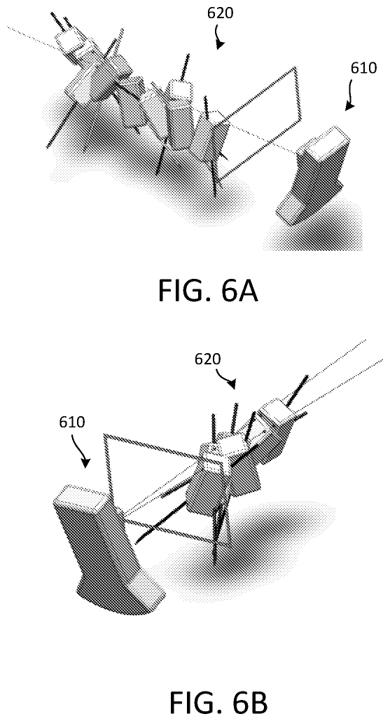

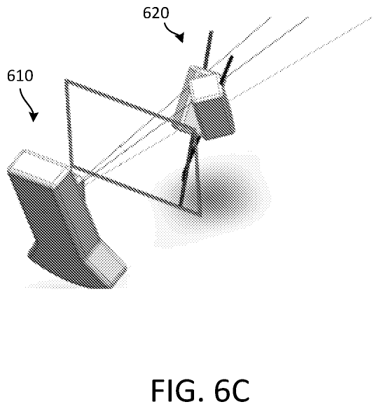

In various embodiments, and with reference to FIGS. 6A, 6B, and 6C, position and orientation ambiguity of the needle guide 620 relative to the probe 610 would result if the system did not have a sufficient number of fiducials. If one fiducial is used, as shown in FIG. 6A, the camera is able to identify the azimuth and elevation position of the fiducial, but not the distance from the camera to the fiducial, nor the orientation of the probe in space. The addition of a second fiducial, as shown in FIG. 6B, further information is provided pertaining to the position and orientation of the needle guide and thereby reduces the number of possible positions of the needle guide, however there is still insufficient information to unambiguously identify the needle guide position. The addition of a third fiducial, as shown in FIG. 6C,), further reduces the number of possible positions of the needle guide. In this case, there are only two possible positions of the needle guide. Although they are quite close to each other and overlap, the difference in orientation means the needles are pointing in different directions, so there is still not enough information to uniquely fix the position. Accordingly, when four fiducials are used, as in the needle guidance system 100 provided and described herein, there is sufficient information to unambiguously resolve the position of the needle guide 120. Given a camera 112 of known geometry and a fixed spacing between fiducials 122, there is only one possible position and orientation of the needle guide 120 that produces the given image of four fiducials. As mentioned above, in various embodiments the fourth fiducial may be replaced by a second camera. That is, three fiducials and two cameras may be used to uniquely fix (e.g., determine) the position of the needle guide. Once the position of the fiducials are known, the position of every other part of the needle guide system (i.e., the tip of the needle and axis) are also known.

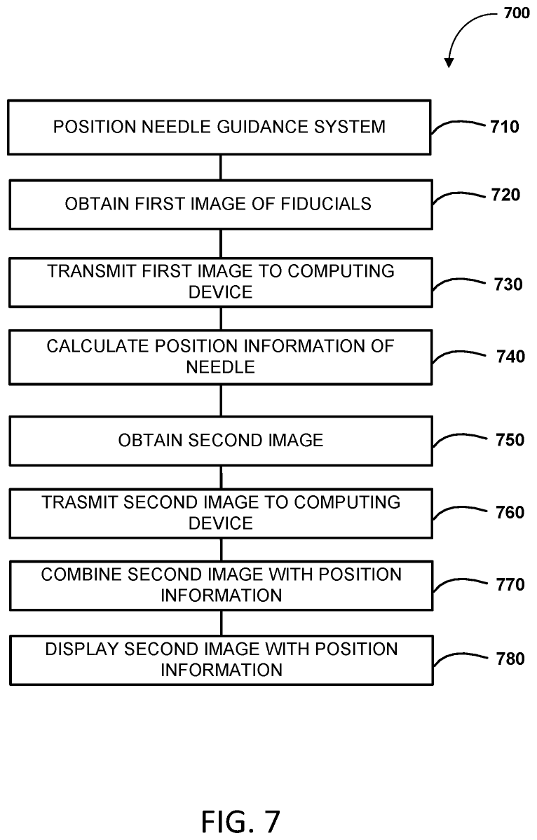

FIG. 7 illustrates a method 700 for providing position information of a needle according to embodiments of the disclosure. At step 710, method 700 comprises positioning the needle guidance system. The needle guidance system may be positioned near or against a body in order to place the needle into the body. The needle guidance system may comprise any of the needle guidance systems described herein. In some embodiments, the needle guidance system comprises a probe comprising a probe transducer and a camera; and a needle guide comprising the needle and a plurality of fiducials. The method 700 further comprises, using the camera to obtain a first image of the plurality of fiducials 720, transmitting the first image to a computing device 730, and using the computing device to calculate the position information of the needle 740. In some embodiments, the position information comprises the position and orientation of the needle relative to the probe. In some embodiments, method 700 further comprises using the probe transducer to obtain a second image 750, transmitting the second image to the computing device 760, and using the computing device to combine the second image with the position information 770, and displaying the second image with the position information on an output device. In some embodiments, the probe comprises an ultrasound probe and the ultrasound probe is positioned against a body. In some embodiments, the computing device is further configured to calculate an anticipated path or trajectory of the needle.

In various embodiments, a method of operating a needle guidance system is provided, including the various operations performed by a controller or other processor. In various embodiments, the first step of the operating method is image acquisition. That is, the first step may be to acquire an image of the needle guide and fiducials using a camera mounted in the ultrasound probe. The camera may be specifically configured to account for the particular needs of the application, including focal distance, depth of focus, field of view and resolution. The camera may be interfaced (e.g., electrically connected) with a controller or other processor to provide the one or more images in a computer-readable format in real time.

The method may further include processing the image. This step may include semantic image segmentation, which refers to identifying pixels in the image associated with the fiducials, separating them from the background and other elements of the images and determining the X and Y coordinates of the fiducials in the coordinate system of the image plane. This step may be accomplished by training a convolutional neural network (CNN) in a multi-target architecture. In various embodiments, the input to the CNN is the numeric array representing the image acquired by the camera and the target output is a set of eight values, representing the X and Y, coordinates for each of the four fiducials. A robust training process may be needed so that this mapping of inputs to outputs can be made regardless of extraneous background noise. The output of this method step may be a vector of the eight coordinates which becomes the input for other steps of the operating method.

The method may further include a 3D spatial resolution step. That is, after the input image has been reduced to a vector of eight values in the coordinate system of the image plane, this spatial resolution step may include creating an algorithm to map this input vector to a set of six values that fully resolve the position and orientation of the needle guide in space. These six values can be thought of as X, Y, Z, roll, pitch and yaw. The relationship between the 8-vector input and the 6-vector output may be a transcendental function of trigonometric functions. A reasonable approximation of this function, with substantial accuracy suitable for this application, may be created with a deep-learning (DL) neural network. A DL neural network may be beneficial for performing this step because the function may be highly non-linear, and in some cases relationships between inputs and outputs may be inverted or cyclical, therefore a simple linear model may not suffice.

This DL neural model can be trained and optimized by creating a training data set in which sets of 6-vector outputs and corresponding 8-vector inputs are calculated from simple geometric relationships. These corresponding inputs and target values are then fed into a multi-input, multi-target DL neural network which is then trained to establish a mapping between them, according to various embodiments. In response to the 6-vector coordinates being determined, the position and orientation of the needle guide is fully resolved and can then be integrated into the image from the ultrasound transducer. Accordingly, the method may include integrating the position of the needle into a useful view for the practitioner to see.

The method may further include an alignment and calibration step. This step may include introducing alignment and calibration factors to accommodate the differences between ideal and real conditions. For example, the function of the camera may be modeled as a flat plane perpendicular to a midline, but in reality, it might have some optical aberration which may be modeled as a section of a sphere, or a torus or more complex shape. Also due to manufacturing variance and other factors, the alignment between the ultrasound image beam and the camera might deviate from nominal. Therefore, the method may include the step of creating a set of algorithms that can identify and correct for these deviations between ideal and real values.

Benefits, other advantages, and solutions to problems have been described herein with regard to specific embodiments. Furthermore, the connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements of the disclosure.

The scope of the disclosure is accordingly to be limited by nothing other than the appended claims, in which reference to an element in the singular is not intended to mean “one and only one” unless explicitly so stated, but rather “one or more.” It is to be understood that unless specifically stated otherwise, references to “a,” “an,” and/or “the” may include one or more than one and that reference to an item in the singular may also include the item in the plural. All ranges and ratio limits disclosed herein may be combined.

Moreover, where a phrase similar to “at least one of A, B, and C” is used in the claims, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C. Different cross-hatching is used throughout the figures to denote different parts but not necessarily to denote the same or different materials.

The steps recited in any of the method or process descriptions may be executed in any order and are not necessarily limited to the order presented. Furthermore, any reference to singular includes plural embodiments, and any reference to more than one component or step may include a singular embodiment or step. Elements and steps in the figures are illustrated for simplicity and clarity and have not necessarily been rendered according to any particular sequence. For example, steps that may be performed concurrently or in different order are illustrated in the figures to help to improve understanding of embodiments of the present disclosure.

Any reference to attached, fixed, connected or the like may include permanent, removable, temporary, partial, full and/or any other possible attachment option. Additionally, any reference to without contact (or similar phrases) may also include reduced contact or minimal contact. Surface shading lines may be used throughout the figures to denote different parts or areas but not necessarily to denote the same or different materials. In some cases, reference coordinates may be specific to each figure.

Systems, methods and apparatus are provided herein. In the detailed description herein, references to “one embodiment,” “an embodiment,” “various embodiments,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element is intended to invoke 35 U.S.C. 112(f) unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

What is claimed is:

1. A needle guidance system comprising:
a probe comprising a probe transducer and a camera; and
a needle guide configured to retain a needle,
wherein the needle guide comprises a plurality of fiducials.

2. The needle guidance system of claim 1, wherein the plurality of fiducials comprises at least four fiducials.

3. The needle guidance system of claim 1, wherein the probe comprises an ultrasound probe.

4. The needle guidance system of claim 3, wherein the probe transducer is an ultrasound transceiver configured to transmit and receive ultrasound.

5. The needle guidance system of claim 1, wherein each fiducial of the plurality of fiducials has a characteristic that is unique from other fiducials of the plurality of fiducials.

6. The needle guidance system of claim 1, wherein each fiducial of the plurality of fiducials comprises a color that is different from other fiducials of the plurality of fiducials.

7. The needle guidance system of claim 1, wherein each fiducial of the plurality of fiducials comprises a shape that is different from other fiducials of the plurality of fiducials.

8. The needle guidance system of claim 1, wherein the system uses a heuristic calculation to distinguish each fiducial from the other fiducials of the plurality of fiducials.

9. The needle guidance system of claim 1, wherein the camera is a wide-angle camera.

10. The needle guidance system of claim 1, wherein the camera comprises two cameras, and wherein the plurality of fiducials comprises three fiducials.

11. The needle guidance system of claim 1, wherein the probe and the needle guide are configured to be manipulated independently of each other.

12. A method of providing position information of a needle, the method comprising:
positioning a needle guidance system, the system comprising:
probe comprising a probe transducer and a camera, and
a needle guide comprising the needle and a plurality of fiducials;
using the camera, obtaining a first image of the plurality of fiducials;
transmitting the first image to a computing device;
using the computing device, calculate the position information of the needle;
using the probe transducer, obtaining a second image;
transmitting the second image to the computing device;
using the computing device, combining the second image with the position information; and
displaying the second image with the position information on an output device.

13. The method of claim 12, wherein the position information comprises the position and orientation of the needle relative to the probe.

14. The method of claim 12, wherein the probe comprises an ultrasound probe, and wherein the ultrasound probe is positioned against a body.

15. The method of claim 14, wherein the probe transducer is an ultrasound transceiver configured to transmit and receive ultrasound.

16. The method of claim 12, wherein the computing device is further configured to calculate a trajectory of the needle.

17. The method of claim 12, wherein the plurality of fiducials comprises at least four fiducials.

18. The method of claim 12, wherein each of the plurality of fiducials has a characteristic that is unique from the other of the plurality of fiducials.

19. The method of claim 12, wherein the camera is a wide-angle camera.

20. The method of claim 12, wherein the camera comprises two cameras, and wherein the plurality of fiducials comprises three fiducials.
